(19) United States
(12) Patent Application Publication
(10) Pub. No.: US 2017/0336381 A1
(43) Pub. Date: Nov. 23, 2017

(54) SENSING OF WATER QUALITY

(71) Applicant: ECO NET LTD., Hod Hasharon (IL)

(72) Inventor: Dani Zeevi, Hod Hasharon (IL)

(21) Appl. No.: 15/537,455

(22) PCT Filed: Oct. 20, 2015
(86) PCT No.: PCT/IB2015/058057

§ 371 Date: Jun. 19, 2017

Related U.S. Application Data
(63) Continuation-in-part of application No. PCT/IB2015/053081, filed on Apr. 28, 2015.
(60) Provisional application No. 62/099,604, filed on Jan. 5, 2015.
(60) Provisional application No. 61/992,236, filed on May 13, 2014.

(51) Int. Cl. G01N 33/18; A01K 63/04; G01V 8/10; G01N 21/51; G01N 29/02

(52) U.S. Cl. CPC G01N 33/1886; G01N 21/51; G01N 29/02; G01V 8/10; A01K 63/04; H04Q 2209/40; H04Q 2209/823; G01N 2291/0228

(57) ABSTRACT

Sensing apparatus (24) includes one or more sensors (26, 28, 46, 48), configured to sense properties of water in which the sensing apparatus floats. A wireless communication interface (42) is coupled to transmit signals indicative of an output of the one or more sensors. An energy storage device (58) is coupled to provide electrical energy to the one or more sensors and to the wireless communication interface. A sealed case (25) contains the one or more sensors, the wireless communication interface, and the energy storage device and has sufficient buoyancy to float in the water.

### CROSS-REFERENCE TO RELATED APPLICATIONS

This application claims the benefit of U.S. Provisional Patent Application 62/099,604, filed Jan. 5, 2015, and is a continuation-in-part of PCT Patent Application PCT/IB2015/053081, filed Apr. 28, 2015, which claims the benefit of U.S. Provisional Patent Application 61/992,236, filed May 13, 2014. All of these related applications are incorporated herein by reference.

### FIELD OF THE INVENTION

The present invention relates generally to sensing and monitoring of fluid properties, and particularly to sensors, systems and methods for monitoring water in a tank.

### BACKGROUND

Aquarium hobbyists invest large amounts of money, time and effort in installing and maintaining their tanks and populating them with fish, coral, plants and décor. Such tanks generally contain a heater or chiller to keep the temperature constant, pumps and filters to preserve water quality, and lights for illumination and plant growth. Conscientious aquarium operators regularly check water quality parameters, such as temperature, pH and ammonia concentration, to ensure that they are in the optimal range, and take corrective action when they are not. All too frequently, however, equipment failures or brief inattention by the operator lead to sudden deterioration in water quality, with catastrophic impact on the fish and other life in the aquarium.

In response to such problems, a number of vendors offer automated aquarium monitoring solutions. For example, Seneye Ltd. (Norwich, Norfolk, UK) offers an electronic device for fish tanks and ponds that monitors temperature, water level, pH, ammonia and other parameters. The device can also be connected via the Internet to a server, which allows the user to view the results using a mobile phone application and provides SMS and e-mail alerts.

As another example, Chinese Utility Model CN 203101372U describes a water quality monitoring system for a fish tank. The system comprises a dissolved oxygen detecting module, a pH value detecting module, a chloride ion detecting module, a temperature detecting module, an AD (Analog/Digital) sampling module, a power supply module, a display module and an alarm module, all connected to a controller module. The system is mainly used for monitoring changes in water quality in a fish tank in real time.

### SUMMARY

Embodiments of the present invention that are described hereinbelow provide novel devices, methods and systems for collecting, processing and providing information with regard to water held in a tank, such as an aquarium.

There is therefore provided, in accordance with an embodiment of the invention, sensing apparatus, including one or more sensors, configured to sense properties of water in which the sensing apparatus floats. A wireless communication interface is coupled to transmit signals indicative of an output of the one or more sensors. An energy storage device is coupled to provide electrical energy to the one or more sensors and to the wireless communication interface. A sealed case contains the one or more sensors, the wireless communication interface, and the energy storage device and has sufficient buoyancy to float in the water.

In a disclosed embodiment, the one or more sensors include an image sensor. Additionally or alternatively, the output of at least one of the sensors is indicative of a quality of the water. Further additionally or alternatively, at least one of the sensors includes an accelerometer and/or an acoustic sensor.

In some embodiments, the wireless communication interface is configured to transmit the signals over the air to a receiver using a short-range radio-frequency (RF) communication protocol. In one embodiment, the signals transmitted by the wireless communication interface include data packets, and the receiver is included in a wireless network access point, which transmits the data packets over a network to a server for analysis of the output.

Typically, the apparatus is configured to float freely in a tank of the water without tethering the case to the tank.

There is also provided, in accordance with an embodiment of the invention, a method for monitoring water in a tank. The method includes deploying in the tank a buoyant sensing device, which is configured to sense properties of water in which the sensing device floats and to transmit over the air, to a wireless receiver, data that are indicative of the sensed properties. The data are received from the wireless receiver via a public communication network and are processed in order to analyze a property of the water. An alert is issued when the property deviates from a predefined normal range.

In some embodiments, the tank includes an aquarium, and issuing the alert includes notifying an operator of the aquarium of a water condition detrimental to fish in the aquarium. In a disclosed embodiment, receiving the data includes receiving one or more images captured by the sensing device.

Alternatively, the tank contains drinking water, and issuing the alert includes notifying an operator of the tank of a water condition detrimental to potability of the water.

In some embodiments, receiving the data includes receiving inputs from multiple sensing devices deployed in tanks in different, respective locations distributed over a geographical area, and the method includes analyzing the received inputs in order to detect macroscopic phenomena extending over the geographical area. In a disclosed embodiment, the sensing devices are configured to sense waves in the water, and analyzing the received inputs includes detecting seismic phenomena responsively to the sensed waves. Additionally or alternatively, analyzing the received inputs includes detecting meteorological phenomena responsively to the received inputs.

There is additionally provided, in accordance with an embodiment of the invention, a monitoring system, including a network interface, which is configured to receive, via a public communication network, data output by multiple buoyant sensing devices, which are deployed in water tanks in different, respective locations distributed over a geographical area and are configured to sense properties of water in which the sensing devices float. A processor is configured to process the received data in order to detect macroscopic phenomena extending over the geographical area.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a system for monitoring water quality in an aquarium, in accordance with an embodiment of the invention;

FIG. 2 is a block diagram that schematically illustrates functional components of a water quality sensing device, in accordance with an embodiment of the invention; and

FIG. 3 is a schematic, pictorial illustration of a system for electronic data collection and processing, in accordance with an embodiment of the invention.

### DETAILED DESCRIPTION OF EMBODIMENTS

The above-mentioned PCT Patent Application PCT/IB2015/053081 describes immersible sensing devices that can be attached to the distal end of a fishing line and transmit signals with respect to the presence or absence of fish in the vicinity, as well as other water quality factors. (The sensing devices are “immersible” in the sense that they operate while partially immersed in a body of water.) These signals can be processed, for example, in order to enhance the angler's fishing experience, as well as sharing information over a network of anglers.

Embodiments of the present invention that are described herein extend the principles of the sorts of sensing devices and systems described in the above-mentioned PCT patent application to monitoring closed tanks of water, such as aquariums and tanks for drinking water. The disclosed embodiments make use of sensing devices, comprising one or more sensors, which are contained in a sealed case having sufficient buoyancy to float in the water. The sensors sense properties of water in which the sensing devices floats, and a wireless communication interface, likewise contained in the sealed case, transmits signals that are indicative of the sensor outputs. The case also contains an energy storage device, such as a primary or rechargeable battery, which provides electrical energy to the sensors and communication interface.

The wireless communication interface transmits the signals over the air to a wireless receiver, typically (although not necessarily) in the form of data packets transmitted to a wireless network access point. The access point or other receiver transmits the data via a public communication network to a server, which processes the received data in order to analyze a property of the water, and issues an alert when the property deviates from a predefined normal range. In embodiments in which the tank comprises an aquarium, for example, the alert can notify an operator of a water condition detrimental to fish in the aquarium. Alternatively, when the tank contains drinking water, the alert can notify an operator of the tank of a deterioration of potability of the water.

Some embodiments of the present invention take advantage of the deployment of multiple sensing devices by different users in water tanks at different, respective locations, distributed over a geographical area, in order to build a data collection and sensing network. The server collects the data from the sensing devices via a wide-area network, such as the Internet. By collating the inputs received from sensing devices at multiple locations, the server can detect macroscopic phenomena extending over the geographical area, for application in environmental monitoring, research and protection, as well as weather monitoring and forecasting, for example. Such monitoring can be conducted in real time or offline.

Additionally or alternatively, when the sensing devices are configured to sense waves in the water (by inertial or acoustic sensing, for example), the server can analyze the inputs received from the distributed sensing devices in order to detect seismic phenomena, and possibly provide early warnings of earthquakes and tsunamis.

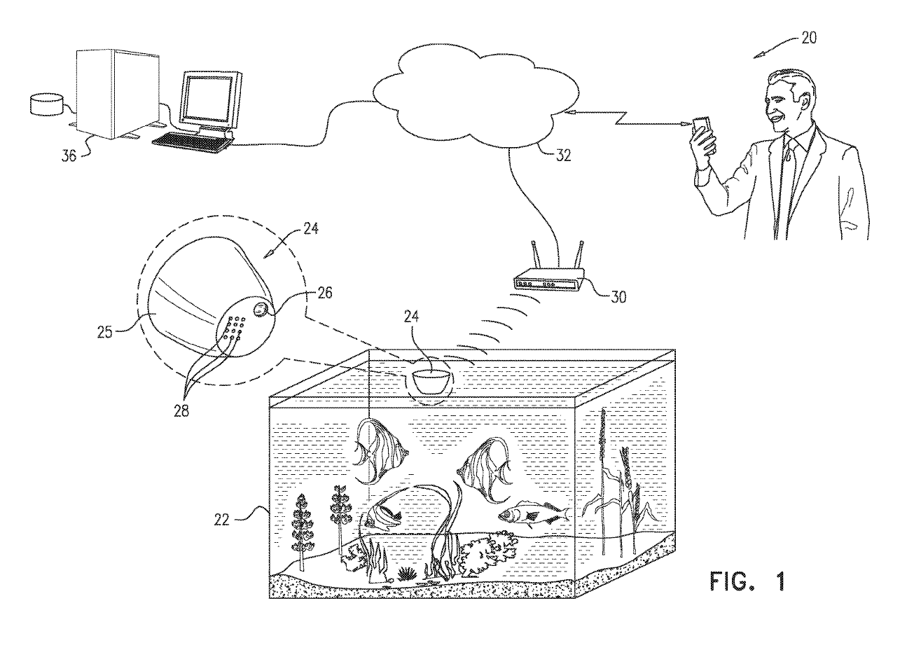

FIG. 1 is a schematic, pictorial illustration of a system 20 for monitoring water quality in an aquarium 22, in accordance with an embodiment of the invention. (As noted earlier, similar systems may be used in monitoring water tanks of other sorts, such as tanks for drinking water that serve residences or other buildings.)

System 20 is built around a floating sensing device 24, having a sealed case 25 with sufficient buoyancy to float in the water in aquarium 22. The case typically comprises a suitable molded plastic material, but may alternatively comprise an inert metal or any other suitable sort of waterproof shell. An outer covering or “skin” (not shown) may be fitted over case 25 for aesthetic purposes, for example to give device 24 the appearance of a floating water plant or other decorative object. Although case 25 in FIG. 1 has a certain geometrical shape, the case may have any other suitable shape, as will be apparent to those skilled in the art. Typically, device 24 is configured to float freely in aquarium 22 and does not require that case 25 be tethered to the aquarium tank or have any external mechanical or electrical connections.

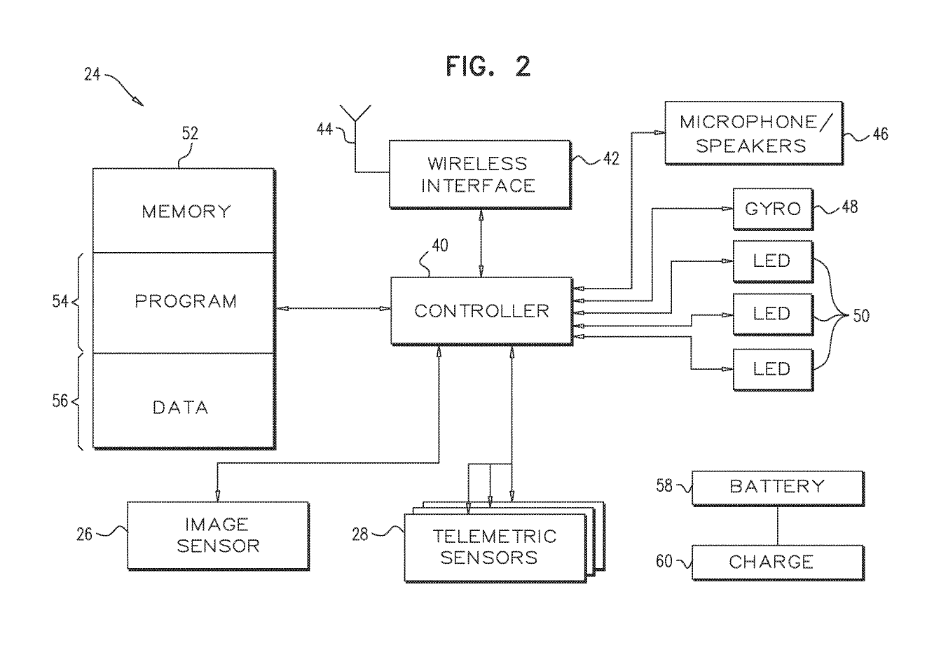

Case 25 contains one or more sensors 26, 28, as well as a wireless communication interface and energy storage device (shown in FIG. 2). Sensors 26, 28 sense properties of the water in which device 24 floats and provide outputs that are indicative of the quality of the water. In the pictured embodiment, sensor 26 is an image sensor with suitable optics (not shown), which captures images below the surface of the water, while sensors 28 comprise other types of water quality sensors. Additionally or alternatively, device 24 may contain acoustic and/or inertial sensors within case 25, as well as other suitable types of sensors depending upon application requirements. Although image sensor 26 is useful in detecting activity (or inactivity) of fish in aquarium 22 and can also be used in measuring properties of the water, such as turbidity, in an alternative embodiment (not shown) device 24 does not include an image sensor.

Device 24 contains a wireless communication interface (shown in the figures that follow), which transmits signals over the air to a wireless receiver 30, such as a wireless network access point. These signals are indicative of the outputs of sensors 26, 28, etc., and typically include a digital still or video output from image sensor 26 and/or telemetric readings from sensors 28. The wireless link may also carry control inputs, configurations and instructions from receiver 30 to device 24. Device 24 and receiver 30 typically communicate over the wireless link using a short-range radio-frequency (RF) communication protocol, such as a Wi-Fi (IEEE 802.11) or Bluetooth protocol. The signals transmitted by the wireless communication interface in device 24 may thus comprise data packets, which receiver 30 forwards over a public wide-area network 32, such as the Internet.

A server 36 receives the data relayed by wireless receiver 30 via network 32 and processes the received data in order to analyze properties of the water in aquarium 22. Server 36 issues reports of water quality, based on this analysis, to client devices 34, such as a fixed or mobile computing device (for example, a smart phone) used by an operator of aquarium 22. These reports typically include alerts that are issued by server 36 when a property of the water in aquarium, such as temperature or a chemical property, or possibly an image-based property, such as movement of the fish, deviates from a predefined normal range and is thus indicative of a water condition detrimental to fish in the aquarium. Upon receiving the alert on client device 34, the operator of aquarium 22 can immediately make any necessary adjustments to the aquarium equipment or replace the water in the aquarium if necessary.

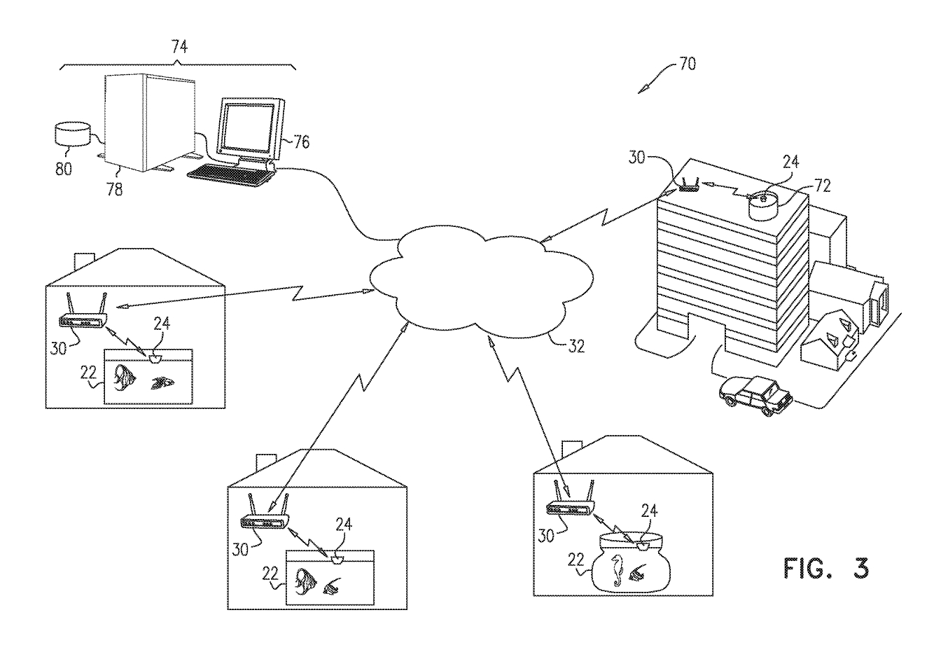

Additionally or alternatively, server 36 collates data received from sensing devices in multiple different aquariums or other water tanks, as described hereinbelow with reference to FIG. 3.

Further additionally or alternatively, client device 34 may receive data from receiver 30 without intervention of server 36. In this case, an application running on client device 34 analyzes the data and provides the desired reports and alerts to the operator.

FIG. 2 is a block diagram that schematically illustrates functional components of sensing device 24, in accordance with an embodiment of the invention. As explained earlier, sensors 28 in device 24 sense parameters used in telemetric monitoring of water quality. The term “water quality” should be broadly understood in this context and in the claims to include any and all characteristics of the water, as well as nearby objects in the water. Thus, sensors 28 may sense, for example, water temperature; pH, salinity, oxygen, and/or other chemical parameters; nearby motion and/or vibration; and/or turbidity. Alternatively or additionally, images captured by image sensor 26 may be analyzed to derive turbidity and other optical qualities of the water, as well as to detect the presence (or absence) of fish and specifically the motion or inactivity of fish that are present. For these purposes, image sensor 26 may receive and sense visible or infrared light, or both.

To detect waves, vibrations or other movement of the water in aquarium 22, device 24 typically comprises an acoustic transducer 46, which is configured as a microphone to sense sonic and/or ultrasonic waves in the water. Additionally or alternatively, device 24 comprises a motion sensor 48, such as an accelerometer or other inertial sensor (commonly referred to as a “gyro”), which generates an output indicative of the motion of the device, and hence of the water in which the device is floating.

Additionally, sensors 28 may comprise air quality, temperature, and barometric sensors (not shown in the figures). Such sensors are typically mounted on the upper side of the device, rather than the lower side as shown in FIG. 1. These sensors are useful both in providing local information to the operator of the water tank and in gathering weather-related information from multiple locations over a wide area for transmission over network 32.

The functions of sensing device 24 are controlled and coordinated by a controller 40, which is typically a single-chip component with suitable interfaces for connection to the other components of device 24. Controller 40 and at least some of the other components shown in FIG. 2 are typically mounted on a rigid or flexible printed circuit board (not shown) inside case 25.

Controller 40 communicates with receiver 30 via a wireless communication interface 42, such as a Wi-Fi or Bluetooth interface, for example, which is connected to an antenna 44 located either inside or on the surface of case 25. A memory 52, comprising non-volatile memory (such as ROM and/or flash memory), and possibly volatile memory (such as RAM), as well, stores program code 54 to be run by controller 40 and data 56 collected by the controller from sensors 26, 28, 46, 48, . . . . Typically, controller 40 digitizes, pre-processes and may even process the outputs of the sensors before transmitting digital signals carrying the data (with or without processing by the controller) to receiver 30 via wireless interface 42. The digital signals make take the form of data packets, and device 24 may have its own Internet Protocol (IP) and/or other network address, which enables receiver 30 to forward the packets directly between device 24 and server 36. Alternatively, communication interface 42 may be configured to transmit the sensor outputs in analog form.

In some embodiments, device 24 comprises one or more light-emitting diodes (LEDs) 50 or other light sources. For example, device 22 may comprise multiple LEDs 50 of different colors, which are used to cast light into the water for purposes of imaging by image sensor 26, as well as water quality sensing. Additionally or alternatively, acoustic transducer 46 may be configured as a speaker to emit sounds or other vibrations for stimulating movement by fish.

Further additionally or alternatively, device 24 may comprise a location sensor, such as a GPS receiver (not shown in the figures). The output of the location sensor can be used to track the current location of device 24 for purposes of large-scale monitoring over a geographical area, as described below.

Controller 40 and the other components of sensing device 24 are powered by a battery 58, which typically holds sufficient charge for at least several days of continuous operation. Battery 58 may be rechargeable via a charging circuit 60, such as a wireless inductive charging circuit. Alternatively or additionally, battery 58 may be a primary battery, storing sufficient energy to run device 24 for months or even years (particularly if image sensing is not required). When the battery runs out, it may be replaced by opening device 24; or the entire device may be replaced. To extend the life of battery 58, the components of device 24 may switch on only when sensors 28 detect that the device is in the water and only periodically thereafter, upon activation either by an internal clock or by a trigger from server 36.

FIG. 3 is a schematic, pictorial illustration of a system 70 for electronic data collection and processing, in accordance with an embodiment of the invention. System 70 collects and processes information transmitted by sensing devices 24 that are deployed in water tanks distributed at different locations over a wide geographical area and are configured to sense properties of water in which the sensing devices float. Sensing devices 24 in this example are deployed in both aquariums 22 and in other sorts of tanks, such as a tank 72 of drinking water belonging to a residential or commercial building.

A server 74 receives and processes information transmitted by sensing devices 24 via receivers 30. Server 74 typically comprises a general-purpose computer, which comprises a processor 78 with a suitable interface 76 to network 32, for communicating with devices 24, and a memory 80. Processor 78 carries out the functions that are described herein under the control of software, which is typically stored in tangible, non-transitory computer-readable media, such as optical, magnetic, or electronic memory media. Server 74 performs the functions of server 36 that were described above, such as analyzing sensor outputs and issuing alerts to the respective operators of aquariums 22 and/or drinking water tank 72, as well as analyzing and detecting macroscopic phenomena, extending over a wide geographical area, as described below.

Server 74 uses various different kinds of data from sensing devices 24 in system 70 for purposes of macroscopic monitoring. For example, server 74 may use vibration and/or other motion-related data transmitted by sensing devices 24, as well as weather-related data, such as temperature and barometric readings. Server 74 may also receive other sorts of data from devices 24, such as GPS-based or other location data. Server 74 correlates the sensor outputs with location data in order to map macroscopic phenomena, such as seismic vibrations and/or changes in weather conditions that correlate over a large number of sensing devices 24 in different locations. On this basis, server 74 can provide alerts when a property associated with the macroscopic phenomena of interest deviates from its predefined normal range, for example, when an increase in wave activity in the water tanks indicates that an earthquake may be imminent, or a drop in barometric pressure indicates that a storm is brewing. Additionally or alternatively, server 74 may provide collated reports of sensing results for purposes of weather forecast or seismological research.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. Sensing apparatus, comprising:
one or more sensors, configured to sense properties of water in which the sensing apparatus floats;
a wireless communication interface coupled to transmit signals indicative of an output of the one or more sensors;
an energy storage device, coupled to provide electrical energy to the one or more sensors and to the wireless communication interface; and
a sealed case, which contains the one or more sensors, the wireless communication interface, and the energy storage device and has sufficient buoyancy to float in the water.

2. The apparatus according to claim 1, wherein the one or more sensors comprise an image sensor.

3. The apparatus according to claim 1, wherein the output of at least one of the sensors is indicative of a quality of the water.

4. The apparatus according to claim 1, wherein at least one of the sensors comprises an accelerometer.

5. The apparatus according to claim 1, wherein at least one of sensors comprises an acoustic sensor.

6. The apparatus according to claim 1, wherein the wireless communication interface is configured to transmit the signals over the air to a receiver using a short-range radio-frequency (RF) communication protocol.

7. The apparatus according to claim 6, wherein the signals transmitted by the wireless communication interface comprise data packets, and wherein the receiver is comprised in a wireless network access point, which transmits the data packets over a network to a server for analysis of the output.

8. The apparatus according to claim 1, wherein the apparatus is configured to float freely in a tank of the water without tethering the case to the tank.

9. A method for monitoring water in a tank, the method comprising:
deploying in the tank a buoyant sensing device, which is configured to sense properties of water in which the sensing device floats and to transmit over the air, to a wireless receiver, data that are indicative of the sensed properties;
receiving the data from the wireless receiver via a public communication network;
processing the received data in order to analyze a property of the water; and
issuing an alert when the property deviates from a predefined normal range.

10. The method according to claim 9, wherein the tank comprises an aquarium, and wherein issuing the alert comprises notifying an operator of the aquarium of a water condition detrimental to fish in the aquarium.

11. The method according to claim 10, wherein receiving the data comprises receiving one or more images captured by the sensing device.

12. The method according to claim 9, wherein the tank contains drinking water, and wherein issuing the alert comprises notifying an operator of the tank of a water condition detrimental to potability of the water.

13. The method according to claim 9, wherein receiving the data comprises receiving inputs from multiple sensing devices deployed in tanks in different, respective locations distributed over a geographical area, and wherein the method comprises analyzing the received inputs in order to detect macroscopic phenomena extending over the geographical area.

14. The method according to claim 13, wherein the sensing devices are configured to sense waves in the water, and wherein analyzing the received inputs comprises detecting seismic phenomena responsively to the sensed waves.

15. The method according to claim 13, wherein analyzing the received inputs comprises detecting meteorological phenomena responsively to the received inputs.

16. A monitoring system, comprising:
a network interface, which is configured to receive, via a public communication network, data output by multiple buoyant sensing devices, which are deployed in water tanks in different, respective locations distributed over a geographical area and are configured to sense properties of water in which the sensing devices float; and
a processor, which is configured to process the received data in order to detect macroscopic phenomena extending over the geographical area.

17. The system according to claim 16, wherein the processor is configured to issue an alert when a property associated with the macroscopic phenomena deviates from a predefined normal range.

18. The system according to claim 16, wherein the sensing devices are configured to sense waves in the water, and wherein the processor is configured to detect seismic phenomena responsively to the sensed waves.

19. The system according to claim 16, wherein the processor is configured to detect meteorological phenomena responsively to the received data.
